(19) United States
(12) Patent Application Publication
(10) Pub. No.: US 2003/0026847 A1
(43) Pub. Date: Feb. 6, 2003

(54) Method of treatment for AIDS

(76) Inventors: Herman W. Lance, Birmingham, AL (US); John M. Mayhall, Birmingham, AL (US)

Correspondence Address: BRADLEY ARANT ROSE & WHITE, LLP, INTELLECTUAL PROPERTY DEPARTMENT-NWJ, 1819 FIFTH AVENUE NORTH, BIRMINGHAM, AL, 35203-2104, US

(21) Appl. No.: 10/114,340

(22) Filed: Apr. 2, 2002

Related U.S. Application Data
(63) Continuation of application No. 09/689,418, filed on Oct. 12, 2000, now abandoned.
Application No. 09/689,418 is a continuation of application No. 09/337,715, filed on Jun. 21, 1999, now abandoned.
Application No. 09/337,715 is a continuation of application No. 09/162,018, filed on Sep. 28, 1998, now abandoned.
Application No. 09/162,018 is a continuation of application No. 08/872,448, filed on Jun. 10, 1997, now abandoned.
Application No. 08/872,448 is a continuation of application No. 08/468,623, filed on Jun. 6, 1995, now abandoned.
Application No. 08/468,623 is a continuation of application No. 08/206,200, filed on Mar. 3, 1994, now abandoned.
Application No. 08/206,200 is a continuation of application No. 07/953,051, filed on Sep. 30, 1992, now abandoned.

(51) Int. Cl. A61K 35/28; A61K 35/78

(52) U.S. Cl. 424/579; 424/725

(57) ABSTRACT

AIDS patients having a CD4+ T-lymphocyte cell count of 200 or less are treated by administration of a therapeutically effective amount of mammalian spleen extract, in combination with a therapeutically effective amount of Eleutherococcus extract.

### FIELD OF THE INVENTION

The present invention relates to the medical field, particularly treatment of AIDS.

### BACKGROUND OF THE INVENTION

HIV disease and AIDS are recognized by the United States Center for Disease Control (CDC) as being distinct disorders. While HIV+ patients test positive for the presence of human immunodeficiency virus (HIV), under the CDC definition, AIDS disease requires a CD4+ T-lymphocyte cell count of 200 or less, in addition to HIV infection. Also, AIDS patients become afflicted with opportunistic infections which generally are not present in non-AIDS, HIV+ patients.

Physicians generally prescribe different methods of treatment for HIV+ patients and AIDS patients. The prior art suggests treatment of HIV disease patients with immune modulators. In contrast, the prior art recommends treatment of AIDS patients with antiviral medications.

One immune modulator which has been suggested for use in treating HIV disease patients is PCM-4™, which includes mammalian spleen extract in combination with Eleutherococcus senticosus maxim extract. This product is sold by Omega™ Nutripharm, Inc. of Birmingham, Ala.

However, PCK-4™ has never been suggested for use in treating AIDS disease, since PCM-4™ has not previously been characterized as an antiviral agent, but instead as an immune modulator recommended for treatment of HIV disease, not AIDS.

Furthermore, there is some controversy as to whether HIV is in fact the etiological agent of AIDS, seer e.g., Duesberg, P. H., Proc. Natl. Acad, Sci. USA 88: 1575-1579 (1991). This is because some patients with symptoms of AIDS disease do not test positive for HIV. Additionally, Duesberg, sutra, reports that there is no controlled epidemiological evidence that HIV is pathogenic in the normal United States population.

There remains an urgent need in the art for new methods for treating AIDS.

### SUMMARY OF THE INVENTION

In accordance with the present invention, a method for treating an AIDS patient having a CD4+ T-lymphocyte cell count of 200 or less, comprises administering to the AIDS patient a therapeutically effective amount of mammalian spleen extract, in combination with a therapeutically effective amount of Eleutherococcus extract.

### DESCRIPTION OF THE PREFERRED EMBODIMENTS

As noted above, an AIDS patient having a CD4+ T-lymphocyte cell count of about 200 or less is treated according to one aspect of the invention by administration of a therapeutically effective amount of mammalian spleen extract, in combination with a therapeutically effective amount of Eleutherococcus extract.

It is believed that the present invention is applicable to both AIDS patients which test positive for HIV infection, and AIDS patients exhibiting AIDS symptoms which do not test positive for HIV.

In accordance with another aspect of the present invention, a method of treating an AIDS patient having an AIDS-related opportunistic infection, an AIDS-related cancer or an AIDS-related dermatological disorder comprises administering to the patient the above-described combination of spleen extract and Eleutherococcus extract.

AIDS-related opportunistic infections can be caused by organisms such as Pneumocystis carinii, Toxoplasma gondii, Cryptosporidium huris, Isospora belli, Candida albicans, Cryptococcus neoformans, Histoplasma capsulatum, Cytomegalovirus, Herpes simplex, Varicella-zoster, Mycobacterium avium-intracellulare, Mycobacterium avium complex, Mycobacterium tuberculosis, and Salmonella.

AIDS-related infections also include oral infections such as thrush or candidiasis.

Cancers which are associated with AIDS include AIDS-related lymphomas (e.g., B-cell lymphoma and T-cell lymphoma) and AIDS-related sarcomas (e.g., Kaposi's sarcoma).

In accordance with still another aspect of the invention, a fetus is protected from HIV infection by it's HIV+ mother by administering to the mother a therapeutically effective amount of the pharmaceutical combination of spleen extract and Eleutherococcus extract.

The pharmaceutical combination of the present invention can be administered in conjunction with other AIDS medications such as AZT and DDI, to reduce the nausea and other side effects thereof.

In preferred embodiments, the spleen extract is an extract from porcine spleen, and contains peptides, polypeptides, amino acids and other materials of less than about 4,000 daltons molecular weight. The porcine spleen extract is believed to include immunoactive pentameric amino acid sequences. The porcine spleen extract also is believed to include Thymosin-beta-9 met, Thymosin-beta-4 and splenin.

In preferred embodiments, the spleen extract is orally administered. Accordingly, the spleen extract advantageously is encapsulated so as to transit the stomach and permit absorption via the duodenum.

In particularly preferred embodiments, the spleen extract is administered as separate first and second dosage forms. In accordance with this embodiment, the first dosage form is primarily comprised of peptides, polypeptides and amino acids of about 1,000-4,000 daltons molecular weight, and the separate second dosage form is primarily comprised of peptides, polypeptides and amino acids of about 1,000-2,000 daltons molecular weight.

In accordance with one aspect of the invention, about 100-600 mg of the first dosage form of spleen extract is administered to the patient on a daily basis, whereas about 50-400 μg of the second dosage form of spleen extract is administered to the patient on an alternating day basis. In preferred embodiments, about 300 μg of the first dosage form of spleen extract is administered to the patient on a daily basis, whereas about 100 μg of the second dosage form of spleen extract is administered to the patient on an alternating day basis.

In particularly preferred embodiments, the first dosage form of spleen extract is configured as 100 mg tablets, one each of which is administered to the patient three times daily with meals. It is particularly preferred that the second dosage form of spleen extract be configured as 100 μg capsules, administered every other day with a meal.

Spleen extract preferably is prepared as follows. Porcine spleens that are suitable for human consumption are selected. Fats are removed from the spleens, and they are subjected to sudden cooling. The spleens are required to be flat and measure 30 to 40 cm in length and 10 to 15 cm in width. The color should be a homogeneous dark red without dark spots. The spleens must be free of any meat or fats attached thereto.

After a maximum of 6 hours later, the spleens are hydrolysed as follows. The spleens are first put through a mincer, sieved and pumped into a bio-fermentator for enzymatic hydrolysis in the presence of the enzyme Tryefin, hydrochloric acid (HCl) pH 8.7 and distilled water. The temperature is raised to 40° C., and the solution is stirred occasionally for 2-3 days. The temperature is monitored, controlled and documented during this period. If there are any differences in temperature over a set limit of 45° C., and it exceeds this temperature for more than one hour, the solution is completely discarded.

During the hydrolysis procedure, fats in the solution are removed constantly, as proper hydrolysis is adversely affected by the presence of fats. The solution then is cooled to room temperature. Large particles in the solution are allowed to settle by gravity, and discarded. This leaves a near-clear solution that is slightly turbid. This supernatant solution then is filtered through a filter pump with polypropylene filter cartridges having a pore size of 1 μm.

The filtered aqueous spleen extract solution is lyophylized and the freeze dried powder is the raw material for the first dosage form tablets described above.

The material for the capsules of the second dosage form described above is prepared as follows. The filtered organ extract is precipitated in the presence of sodium chloride to separate high molecular weight materials. The supernatant is filtered again and extracted with 91% phenol. This step removes viral and other contaminated or infectious material.

The phenol extract then is put through column chromatography to isolate an active fraction containing 1,000-2,000 dalton molecular weight peptides, polypeptides and amino acids for the capsules. This fraction is then treated with phenol which is later removed using ether, and the active fraction in aqueous solution is provided. The active fraction then is lyophylized for loading into capsules.

As noted above, in addition to administration of the mammalian spleen extract, the present invention utilizes Eleutherococcus extract. In preferred embodiments, the Eleutherococcus extract comprises a protic extract of Eleutherococcus senticosus maxim (family Araliacae), most preferably an ethanol-water extract thereof. This Eleutherococcus extract is believed to contain eleutherosides A, B, B₁, C, D and E, including daucosterol.

In preferred embodiments, the Eleutherococcus extract is administered on a daily basis, in an amount equivalent to about 0.5-7 ml of an about 27.5% by volume ethanol in water extract of Eleutherococcus.

In particularly preferred embodiments, an equivalent of about 3.3 ml of the Eleutherococcus extract is administered to the patient on a daily basis. The Eleutherococcus extract can be taken as a single dose daily. Alternatively, and preferrably, an equivalent of about 2.2 ml of the Eleutherococcus extract is administered to the patient before breakfast daily, and an equivalent of about 1.1 ml of the Eleutherococcus extract is administered to the patient daily at lunch.

The preferred process for preparing the Eleutherococcus extract is as follows.

Eleutherococcus senticosus maxim root is dug up, shaken free of dirt and sand, and washed with cold flowing water. After this, the root is dried by baking for one hour at 80° C. After this baking process, the roots are allowed to continue drying in a vacuum drier at 60° C. or in a warm, dry and well-ventilated location.

After this preparation, roots are selected based on the following requirements: the roots are whole or in small parts, the bark on the outside is a yellow-brown color and internally the roots are white. The outside bark is wrinkled, indicating that it is dried properly. Breaking the root open reveals a white, slightly fibrous, strongly scented aromatic characteristic. The taste is spicy and slightly bitter.

The thus prepared and dried Eleutherococcus roots are ground to a medium-coarse powder. This powder is mixed with a protic solvent such as 40% ethanol (60% water) by volume for the extraction (1 kg powder per liter of 40% ethanol) and then is heated (State Pharmacopeia of the USSR, page 522). The extraction can be accomplished with other protic solutions, such as water or alcohol-water solutions, for example 10-100% ethanol solutions with water.

The resulting extract is a darkish brown fluid, transparent, with a bitter taste and specific color. This extract has a dry residue of not less than 6% (Pharmacopeia, supra, p. 253), and an alcohol content of not less than 33% (Pharmacopeia, supra, p. 813). When provided as a component of PCM-4™, this extract is diluted with water to an ethanol content of about 27.5%.

PCM-4™ further includes the previously described spleen extract in both tablet and capsule form. The tablets contain 100 mg of the above-described first dosage form of spleen extract. The capsules contain 100 μg of the above-described second dosage form of spleen extract.

The invention is further illustrated by the following examples, which are not intended to be limiting.

### EXAMPLE 1

An AIDS patient having a T-cell count of 112 was treated with PCM-4™ for six weeks with the following regimen: one 100 mg tablet of the first dosage form of spleen extract three times daily with meals, one 100 μg capsule of the second dosage form of spleen extract every other day with a meal, 2.2 ml Eleutherococcus extract as described above before breakfast and 1.1 ml patient's T-cell count had risen to 362, a 223% increase.

### EXAMPLE 2

### The Use of PCK-4™ in the Treatment of AIDS in Uganda

Twenty-seven AIDS patients were studied, and out of those cases, unfortunately three patients died too soon after initiation of the study, and another three died after completing two weeks of the study. There were three defaulters. Two files are not available for analysis, although patients completed the course. An additional three patients were falsely diagnosed to be HIV+.

The patients were treated according to the regimen of Example 1. None of the patients on PCR-4™ required a dosage change or discontinuation of medicine resulting from side effects.

Of this study, the following cases were available for analysis.

The first case was a 21-year-old female, who had symptoms of extensive oral thrush, high-grade fevers, fatigue, poor appetite, and periodic diarrhea. Seven months prior to the study, she had a history of herpes zoster. She was treated with PCM-4™, and after two weeks of the treatment, her appetite increased, fever was controlled, and the frequency of diarrhea decreased. The thrush also cleared (she was on Nizerol at the same time). The arthropathy also improved. Treatment was discontinued after eight weeks. The patient's health declined. She developed pneumonia that was controlled by X-Pen and Septrin. As the pneumonia cleared she developed UTI, which was treated by antibiotics. PCM-4™ was later reintroduced to the patient, improving the patient's condition slightly. Several days later, she developed deep jaundice and hepatic encephalopathy and later died.

The second case was a 24-year-old female. She had a persistent cough, periodic diarrhea, skin rashes, and weight loss. After starting treatment, she gained 2 kg., her diarrhea subsided, and, in the sixth to eighth week, the skin rash disappeared.

The third patient was a 26-year-old male who started the study after having been diagnosed with PTB. He was being treated with STH300. He had oral thrush, consolidation lung posteriority, and generalized skin rashes. The patient responded well to the PCM-4™. He gained 4 kg., his diarrhea subsided, and the cough and consolidated lungs improved.

The fourth case was a 35-year-old male. He was experiencing weight loss, painful cervical lymph nodes, and high-grade fevers. Cervical lymph node aspirate contained tubercle bacilli. The patient was receiving antituberculosis therapy, and a week after he began this treatment, he was started on PCM-4™. Since then, the painful lymph nodes have disappeared and the fever has gone down, but he has not gained weight; instead, he has lost 2 kg. Otherwise, he has a strong appetite.

Case five was a 28-year-old male. This patient had symptoms of multiple nodules on his palate and herpes zoster. The patient did not have any other symptoms. He gained 2 kg. while receiving the PCM-4™ treatment, and there was no further growth in his Kaposi's sarcoma lesions. He now works daily as a shopkeeper.

Case six was a 23-year-old female who had symptoms of periodic diarrhea, an itchy rash, sores in her mouth, painful lymph adenitis in the cervical area, and oral and vaginal candidiasis. The patient defaulted after one week after being convinced by friends to do so. However, her condition worsened and she rejoined the study. Her diarrhea disappeared, her weight loss reversed (she gains 8 kg.), and candidiasis cleared. Her skin is now almost normal. This patient improved a lot.

Case seven was a 25-year-old male who had symptoms of right-sided chest pains, painful swelling in his toes, and recurrent multiple abscesses (pyomyositis). In addition to this, the patient had weight loss. After receiving treatment, the patient gained 2 kg., the pyomyositis was totally healed, and he no longer had any pain in his toes.

Case eight was a 28-year-old female who had symptoms of oral thrush, weight loss, diarrhea, a persistent cough for two months, and vomiting. The patient made significant improvement. Her appetite increased. She gained 0.75 kg. The diarrhea and vomiting decreased and the general well-being improved. However, the patient developed social problems when friends and relatives chased her out of her home. The patient died suddenly. The cause of death could have been dehydration or neglect.

Case nine was a 30-year-old male. He had no symptoms, but he gained 2 kg. while taking the treatment.

Case ten was a 45-year-old male. The patient had preterminal renal insufficiency with bilateral small kidney, renal hypertension, and HIV infection with decreased T-4 lymphocytes. He had been discharged in a German hospital as a preterminal case. He also had severe fatigue and a scaly skin rash. He gained 3 kg. on treatment, and his skin regained its moisture. He had been bed-ridden, but later resumed his regular activities with stable renal function.

Case 11 had a skin rash, repeated fevers, general body weakness, and discharge from the ears. His symptoms disappeared, and he gained 105 kg. after six weeks of the PCM-4™ treatment.

Case 12 had general malaise, hyperpigmented, shiny skin and weight loss. The patient improved on PCM-4™, gaining 2.5 kg., and the rash greatly improved.

Case 13 had asymptomatic HIV infection. The patient gained 0.5 kg. while on the treatment.

Case 14 had asymptomatic HIV infection.

Case 15 was a 35-year-old male showing symptoms such as skin rashes, malaise, and recurrent fever. He was diagnosed HIV+ (not certified) at Kamuri and began taking Kemron. However, he stopped taking the drug after two months. He then began taking PCM-4™ and his symptoms reversed at a very fast rate. He gained 7 kg. after eight weeks on the treatment, but in the sixth week, he tested negative on two occasions. PCM-4™ was discontinued. It is possible that PCM-4™ was administered under a false diagnosis or the patient seroconverted.

Case 16 actually is two cases who were the wives of Case 14. They were mistakenly put on PCM-4™ without an HIV test. When tested later, neither the man nor either of the women showed HIV antibodies. However, they gained 2 and 4 kg. respectively.

Case 17 had PUO for one year and was grossly wasted. Also, he was started on PMC-4™ without HIV serology. He later tested negative. PCM-4™ was discontinued in the sixth week. He gained 2 kg. and his fever disappeared.

### EXAMPLE 3

PCM-4™ is administered according to the regimen described in Example 1 to AIDS patients having AIDS-related opportunistic infections, AIDS-related cancer or AIDS-related dermatological disorders so as to reduce the symptoms associated therewith.

### EXAMPLE 4

An HIV-positive pregnant woman is treated with PCM-4™ according to the regimen described in Example 1 throughout her pregnancy, resulting in delivery of an HIV-negative child.

1. A method of treating an AIDS patient having a CD4+ T-lymphocyte cell count of 200 or less, comprising administering to said patient a therapeutically effective amount of mammalian spleen extract, in combination with a therapeutically effective amount of Eleutherococcus extract.

2. The method of claim 1 wherein the spleen extract is an extract from porcine spleen.

3. The method of claim 2 wherein said spleen extract contains peptides, polypeptides and amino acids of less than about 4,000 daltons molecular weight.

4. The method of claim 3 wherein said spleen extract includes immunoactive pentameric amino acid sequences.

5. The method of claim 3 wherein said spleen extract includes Thymosin-beta-9 met, Thymosin-beta-4 and splenin.

6. The method of claim 3 wherein said Eleutherococcus extract comprises a protic extract of Eleutherococcus senticosus maxim.

7. The method of claim 6 wherein said protic extract comprises an ethanol-water extract.

8. The method of claim 6 wherein said Eleutherococcus extract includes eleutherosides A, B, B₁, C, D and E.

9. The method of claim 6 wherein said Eleutherococcus extract includes daucosterol.

10. The method of claim 6 wherein said spleen extract includes a first dosage form containing primarily peptides, polypeptides and amino acids of about 1,000-4,000 daltons molecular weight, and a second dosage form containing primarily peptides, polypeptides and amino acids of about 1,000-2,000 daltons molecular weight.

11. The method of claim 10 wherein about 100-600 mg of said first dosage form is administered to said patient on a daily basis, about 50-400 μg of said second dosage form is administered to said patient on an alternating day basis, and an amount of said Eleutherococcus extract is administered to said patient on a daily basis which is equivalent to about 0.5-7 ml of an about 27.5% by volume ethanol in water extract of Eleutherococcus.

12. The method of claim 11 wherein about 300 mg of said first dosage form is administered to said patient on a daily basis, about 100 μg of said second dosage form is administered to said patient on an alternating day basis, and an equivalent of about 3.3 ml of said extract of Eleutherococcus is administered to said patient on a daily basis.

13. The method of claim 12 wherein about 100 mg of said first dosage form is administered to said patient three times daily, an equivalent of about 2.2 ml of said extract of Eleutherococcus is administered to said patient before breakfast daily, and an equivalent of about 1.1 ml of said extract of Eleutherococcus is administered to said patient daily at lunch.

14. A method of treating an AIDS patient having an AIDS-related opportunistic infection, an AIDS-related cancer or an AIDS-related dermatological disorder, comprising administering to said patient a therapeutically effective amount of a mammalian spleen extract, in combination with a therapeutically effective amount of Eleutherococcus extract.

15. The method of claim 14 wherein the spleen extract is an extract from porcine spleen.

16. The method of claim 15 wherein said spleen extract contains peptides, polypeptides and amino acids of less than about 4,000 daltons molecular weight.

17. The method of claim 16 wherein said spleen extract includes immunoactive pentameric amino acid sequences.

18. The method of claim 16 wherein said spleen extract includes Thymosin-beta-9 met, Thymosin-beta-4 and splenin.

19. The method of claim 16 wherein said Eleutherococcus extract comprises a protic extract of Eleutherococcus senticosus maxim.

20. The method of claim 16 wherein said protic extract comprises an ethanol-water extract.

21. The method of claim 19 wherein said Eleutherococcus extract includes eleutherosides A, B, B₁, Cr D and E.

22. The method of claim 6 wherein said Ekeutherococcus extract includes daucosterol.

23. The method of claim 19 wherein said spleen extract includes a first dosage form containing primarily peptides, polypeptides and amino acids of about 1,000-4,000 daltons molecular weight, and a second dosage form containing primarily peptides, polypeptides and amino acids of about 1,000-2,000 daltons molecular weight.

24. The method of claim 23 wherein about 100-600 mg of said first dosage form is administered to said patient on a daily basis, about 50-400 μg of said second dosage form is administered to said patient on an alternating day basis, and an amount of said Eleutherococcus extract is administered to said patient on a daily basis which is equivalent to about 0.5-7 ml of an about 27.5% by volume ethanol in water extract of Eleutherococcus.

25. The method of claim 24 wherein about 300 mg of said first dosage form is administered to said patient on a daily basis, about 100 μg of said second dosage form is administered to said patient on an alternating day basis, and an equivalent of about 3.3 ml of said extract of Eleutherococcus is administered to said patient on a daily basis.

26. The method of claim 25 wherein about 100 mg of said first dosage form is administered to said patient three times daily, an equivalent of about 2.2 ml of said extract of Eleutherococcus is administered to said patient before breakfast daily, and an equivalent of about 1.1 ml of said extract of Eleutherococcus is administered to said patient daily at lunch.

27. The method of claim 14 wherein said AIDS-related infection is an infection by Pneumocystis carinii, Toxoplasma gondii, Cryptosporidium huris, Isospora belli, Candida albicans, Cryptococcus neoformans, Histoplasma capsulatum, Cytomegalovirus, Herpes simplex, Varicella-zoster, Mycobacterium avium-intracellulare, Mycobacterium avium complex, Mycobacterium tuberculosis, and Salmonella.

28. The method of claim 14 wherein said AIDS-related cancer is an AIDS-related lymphoma or sarcoma.

29. The method of claim 28 wherein said AIDS-related cancer is B-cell lymphoma, Kaposi's sarcoma or T-cell lymphoma.

30. The method of claim 14 wherein said AIDS-related infection is an oral infection.

31. The method of claim 30 wherein said oral infection is thrush or candidiasis.

32. A method of protecting a fetus from HIV infection by its mother comprising administering to an expectant mother infected with HIV, a therapeutically effective amount of mammalian spleen extract, in combination with a therapeutically effective amount of Eleutherococcus extracts so as to protect said fetus from HIV infection.
